(19) United States
(12) Patent Application Publication
(10) Pub. No.: US 2005/0245962 A1
(43) Pub. Date: Nov. 3, 2005

(54) Catheter with removable wire lumen segment

(75) Inventors: Daniel O. Adams, Long Lake, MN (US); Richard S. Kusleika, Eden Prairie, MN (US)

Correspondence Address: POPOVICH, WILES & O'CONNELL, PA;650 THIRD AVENUE SOUTH, SUITE 600, MINNEAPOLIS, MN, 55402, US

(73) Assignee: ev3 Inc.

(21) Appl. No.: 11/173,448

(22) Filed: Jul. 1, 2005

Related U.S. Application Data
(63) Continuation of application No. 10/460,750, filed on Jun. 12, 2003.

(51) Int. Cl. A61M 29/00

(52) U.S. Cl. 606/194; 623/1.11; 604/103.04

(57) ABSTRACT

The invention provides a catheter for use in combination with an elongate support member. The catheter comprises an elongate body having a proximal portion, a proximal end, a distal portion, a distal end, and a main shaft; an element disposed on the distal portion of the elongate body, the element being an interventional element or a delivery element for delivery of an interventional element; at least one lumen dimensioned to receive the elongate support member; and a tube wall disposed about the lumen. The tube wall has at least one removable segment disposed on the proximal portion of the elongate body and at least one non-removable segment disposed on the distal portion of the elongate body, the removable segment is tubular or generally tubular having a partial circular circumferential cross section, and the transverse cross-sectional area of the removable segment does not comprise the entire transverse cross-sectional area of the catheter.

This application is a continuation of application Ser. No. 10/460,750, filed Jun. 12, 2003, the contents of which are hereby incorporated herein by reference.

### FIELD OF THE INVENTION

This invention relates to devices used in a blood vessel or other lumen in a patient's body. In particular, the present invention relates to catheters having a removable wire lumen segment or segments.

### BACKGROUND OF THE INVENTION

Coronary vessels, partially occluded by plaque, may become totally occluded by thrombus or blood clot causing myocardial infarction, angina, and other conditions. Carotid, renal, peripheral, and other blood vessels can also be restrictive to blood flow and require treatment. A number of medical procedures have been developed to allow for the removal or displacement (dilation) of plaque or thrombus from vessel walls to open a channel to restore blood flow and minimize the risk of myocardial infarction. For example, atherectomy or thrombectomy devices can be used to remove atheroma or thrombus. In cases where infusion of drugs or aspiration of thrombus may be desired, infusion or aspiration catheters can be placed near the treatment site to infuse or aspirate. In cases where the treatment device can be reasonably expected to shed emboli, embolic protection devices can be placed near the treatment site to capture and remove emboli. In other cases, a stent is placed at the treatment site. Both embolic protection devices and stents can be placed in the treatment site using delivery catheters.

In percutaneous transluminal coronary angioplasty (PTCA), a guide wire and guide catheter are inserted into the femoral artery of a patient near the groin, advanced through the artery, over the aortic arch, and into a coronary artery. An inflatable balloon is then advanced into the coronary artery, across a stenosis or blockage, and the balloon inflated to dilate the blockage and open a flow channel through the partially blocked vessel region. One or more stents may also be placed across the dilated region or regions to structurally maintain the open vessel. Balloon expandable stents are crimped onto a balloon in the deflated state and delivered to the lesion site. Balloon expansion expands the stent against the lesion and arterial wall.

In most forms of PTCA, the dilatation catheter is guided into position through the patient's arteries utilizing a very small diameter highly torqueable but flexible guide wire. The distal end of the guide wire is extremely flexible and may be formed as a coil of very small diameter wire over a tapered core wire. This construction enables the cardiac physician to direct the guide wire along the branched and convoluted arterial pathway as the guide wire is advanced to the lesion at the target site. Once the guide wire is positioned across the lesion, an appropriately sized dilatation balloon catheter is advanced over-the-wire by sliding the tubular lumen of the catheter over the guide wire from its proximal end to its distal end. Typically the guide wire used for an over-the-wire PTCA balloon catheter is 300 to 320 cm in length. At this point in the procedure the dilatation balloon is in a deflated configuration having a minimal cross-sectional diameter which facilitates its positioning across the lesion prior to inflation. At various times throughout the procedure radiopaque dyes are injected into the artery to enable the cardiac physician to directly visualize the positioning of the catheter within the target vascular pathway on a fluoroscope.

Dilatation catheter designs other than those of the over-the-wire type have been developed. For example, fixed-wire dilatation catheters incorporating an internally fixed guide wire or stiffening element have been utilized with some success. These fixed-wire designs are smaller in diameter than their over-the-wire counterparts because a single balloon inflation lumen is also used to contain the fixed guide wire. As a result, these designs are quite maneuverable and relatively easy to position. However, with a fixed-wire catheter design, access to the target site over a guide wire is lost when removing or exchanging the catheter.

Another alternative catheter design is the monorail or rapid exchange type such as that disclosed in U.S. Pat. No. 4,762,129, issued Aug. 9, 1988, to Bonzel. This catheter design utilizes a conventional inflation lumen plus a relatively short parallel guiding or through lumen located at its distal end and passing through the dilatation balloon. Guide wires used with PTCA balloon catheters are typically 175 cm in length and are much easier to keep within the sterile operating field than 300 to 320 cm guide wires. This design enables the short externally accessible rapid exchange guide wire lumen to be threaded over the proximal end of a pre-positioned guide wire without the need for long guide wires.

Highly tortuous anatomies or chronic total occlusions require the physician to push hard to advance a catheter over a guide wire. For these types of situations, over-the-wire catheters provide superior wire support as compared to that offered by rapid exchange catheters. However, it is difficult to keep long guide wires within a sterile operating field and the physician's arms are not long enough to hold the guide wire steady near its proximal end while advancing the catheter from a position near the patient's vascular access site. Generally an assistant is required to handle these long guide wires. Rapid exchange catheters and the associated short guide wires can be easily handled by one physician alone and are generally preferred in the market today. However, they do not offer the exceptional support characteristic of over-the-wire catheter designs.

Another catheter design is disclosed in U.S. Pat. No. 4,988,356, issued Jan. 29, 1991, to Crittenden et al. This catheter and guide wire exchange system utilizes a connector fitting mounted on the proximal end of the catheter in conjunction with a longitudinally extending slit in the catheter shaft extending distally from the fitting along the length of the catheter guide wire lumen. A guide member mounted on the fitting directs the guide wire through the slit and into or out of the guide wire lumen in response to relative movement of the guide wire or catheter.

Another catheter design is described in U.S. Pat. No. 5,195,978, issued Mar. 23, 1993, to Schiffer. This catheter has one or more breakaway segments for progressively exposing the guide wire from the proximal end toward the distal end of the catheter. The breakaway element may be formed as a longitudinally aligned pull strip provided in the guide wire lumen or as one or more linearly arrayed tubular breakaway segments in the catheter shaft or as a combination of both features. The linearly arrayed tubular breakaway segments encompass the entire circumferential cross section of the catheter.

A need in the art remains for a catheter which can be used as an over-the-wire catheter and can be easily converted to a rapid exchange catheter before or during an intravascular procedure.

### SUMMARY OF THE INVENTION

The invention provides a catheter for use in combination with an elongate support member. The catheter comprises an elongate body having a proximal portion, a proximal end, a distal portion, a distal end, and a main shaft; an element disposed on the distal portion of the elongate body, the element being an interventional element or a delivery element for delivery of an interventional element; at least one lumen dimensioned to receive the elongate support member; and a tube wall disposed about the lumen. The tube wall has at least one removable segment disposed on the proximal portion of the elongate body and at least one non-removable segment disposed on the distal portion of the elongate body, the removable segment is tubular or generally tubular having a partial circular circumferential cross section, and the transverse cross-sectional area of the removable segment does not comprise the entire transverse cross-sectional area of the catheter.

It is to be understood that both the foregoing general description and the following detailed description are exemplary and explanatory and are intended to provide further explanation of the invention as claimed.

### BRIEF DESCRIPTION OF THE DRAWINGS

FIG. 1A is a side view and FIG. 1B is a transverse, cross-sectional view of a balloon catheter of the invention. FIG. 1C is a cross-sectional view of a portion of another balloon catheter of the invention.

FIG. 2 is a side view of the balloon catheter of FIGS. 1A and 1B after the removable wire lumen segment has been removed.

FIG. 3A is a side view and FIG. 3B is a transverse, cross-sectional view of a balloon catheter of the invention. FIG. 3C is a perspective view of the catheter as the removable wire lumen segment is being removed. FIG. 3D is a side view of a portion of another balloon catheter of the invention.

FIGS. 4A and 4C are side views of a catheter of the invention showing a guide wire lumen manifold that can be clipped into a holder on the inflation port. FIG. 4B is a transverse, cross-sectional view of the catheter.

FIGS. 5A and 5B are side views of a catheter of the invention. FIGS. 5C to 5E are cross-sectional views of alternative embodiments of the removable wire lumen segment. FIGS. 5F to 5J are cross-sectional views of alternative embodiments of the catheter of the invention.

FIG. 6A is a side view and FIG. 6B is a transverse, cross-sectional view of an infusion/dye-injection/suction catheter of the invention. FIG. 6C is a side view of a portion of a catheter of the invention.

FIGS. 7A to 7C are cross-sectional views of alternative embodiments of the balloon catheter of FIG. 3. FIGS. 7D to 7F are cross-sectional views of alternative embodiments of a catheter of the invention.

FIG. 8 is a side view of a stent delivery catheter of the invention.

FIG. 9 is a side view of another stent delivery catheter of the invention.

FIG. 10 is a side view of an embolic protection device delivery catheter of the invention.

### DETAILED DESCRIPTION OF THE PREFERRED EMBODIMENTS

The terms “distal” and “proximal” as used herein refer to the relative position of the guide wire and catheters in a lumen. The most “proximal” point of the catheter is the end of the catheter extending outside the body closest to the physician. The most “distal” point of the catheter is the end of the catheter placed farthest into a body lumen from the entrance site.

The invention provides a catheter for use in combination with an elongate support member. The catheter comprises an elongate body having a proximal portion, a proximal end, a distal portion, a distal end, and a main shaft; an element disposed on the distal portion of the elongate body, the element being an interventional element or a delivery element for delivery of an interventional element; at least one lumen dimensioned to receive the elongate support member; and a tube wall disposed about the lumen. The tube wall has at least one removable segment disposed on the proximal portion of the elongate body and at least one non-removable segment disposed on the distal portion of the elongate body, the removable segment is tubular (full circumferential cross section) or generally tubular having a partial circular circumferential cross section, and the transverse cross-sectional area of the removable segment does not comprise the entire transverse cross-sectional area of the catheter.

This invention applies to any catheter used in conjunction with a guide wire or elongate support member for delivery. The concept is universal. Balloon catheters and stent delivery catheters with or without a balloon are typical catheters to which the invention can be applied. The concept can also be applied to percutaneous delivery and recovery catheters for embolic protection devices, atrial appendage occlusion devices, mitral valve remodeling devices, and the like.

Preferably, the catheter is made with a lumen such as for balloon inflation or other function that is the primary shaft of the device. It preferably includes a full length wire lumen that allows a proximal portion to be completely removed by the user for catheter use in a rapid exchange fashion. The distal portion of the guide wire lumen could also be coaxial or dual lumen as well known in rapid exchange designs.

The invention provides a combination over-the-wire and rapid exchange catheter that has a wire lumen that can be cut, torn away or separated by the operator prior to use, during use, or left in place. In one embodiment, the catheter is a dual lumen (side by side) extrusion with one lumen for inflation/deflation of the balloon and the other for enclosing the guide wire. A balloon is in communication with the inflation lumen.

Another embodiment places a hypotube into the inflation lumen for stiffness or kink resistance or both, or the hypotube comprises the inflation lumen entirely. Alternatively, one or more wires could be placed in the same lumen or molded into the wall of the inflation lumen during extrusion to provide stiffness, kink resistance, or both. One or both alternatives could be used to provide lateral bending resistance, lateral kink resistance, axial stiffness, and compression resistance during removal of the wire lumen.

A catheter of the invention can be loaded onto an elongate support member or guide wire. A typical guide wire or elongate support member is about 0.25 to 0.9 mm in diameter and ranges from 80 cm to 320 cm in length and has a floppy tip at the distal end. The guide wire may be an over-the-wire length guide wire having a frangible joint such that the guide wire can be shortened to a rapid exchange length as described in U.S. 2002/0133092, “Wire Convertible From Over-the-Wire Length to Rapid Exchange Length”, which is hereby incorporated by reference in its entirety herein.

Percutaneous methods of introducing guide wires and catheters and the methods for the removal of such devices from vessels are well known in the art of endovascular procedures. In a typical coronary procedure, the elongate support member and balloon catheter are loaded into a guide catheter and moved into the vessel and through the guide catheter to the treatment site. This is done typically by advancing a first, or introduction guide wire, through the femoral artery to the ascending aorta. A guide catheter is advanced over the guide wire, positioned in a coronary artery ostium, and the introduction guide wire removed. Then a coronary guide wire is advanced through the guide catheter to the coronary artery lesion and across the lesion. Next the balloon catheter or other functional device is advanced down and over the coronary guide wire within the guide catheter to the region of interest. The balloon catheter or other functional device can then be advanced such that the balloon is situated in the lesion to be dilated, and the balloon then inflated. The catheter may then be removed by holding the guide wire in place while pulling back the catheter from the patient's body.

The components of the catheters of the invention are made from biocompatible materials such as metals or polymeric materials. If necessary, these metals or polymeric materials can be treated to impart biocompatibility by various surface treatments, as known in the art. Suitable materials include stainless steel, titanium and its alloys, cobalt-chromium-nickel-molybdenum-iron alloy (commercially available under the trade designation ELGILOY™), carbon fiber and its composites, and polymers such as liquid crystal polymers, polyetheretherketone (PEEK), polyimide, polyester, high density polyethylene, PEBAX, various nylons, and the like. A shape memory or superelastic material such as nitinol or shape memory polymer is also suitable. When wire is used, the wire is selected on the basis of the characteristic desired, i.e., stiffness or flexibility, and the properties can depend upon both the diameter of the wire and its cross-sectional shape. The size, thickness, and composition of materials are selected for their ability to perform as desired as well as their biocompatibility. It is to be understood that these design elements are known to one of skill in the art.

The material comprising the catheter is preferably at least partially radiopaque. This material can be made radiopaque by plating, or by using core wires, tracer wires, or fillers that have good X-ray absorption characteristics compared to the human body. Marker bands comprised of generally tubular radiopaque metals may be attached to the catheter.

The tip of the catheter may be a generally softer material so as to help prevent damage to a vessel wall as the tip is advanced through the vasculature. Softer materials such as PEBAX, nylon, rubbers, urethane, silicone, ethylene vinyl acetate, and the like may be attached to the catheter by adhesives, overmolding, heat bonding, solvent bonding, and other techniques known in the art.

The various embodiments of the invention will now be described in connection with the drawing figures. It should be understood that for purposes of better describing the invention, the drawings have not been made to scale. Further, some of the figures include enlarged or distorted portions for the purpose of showing features that would not otherwise be apparent.

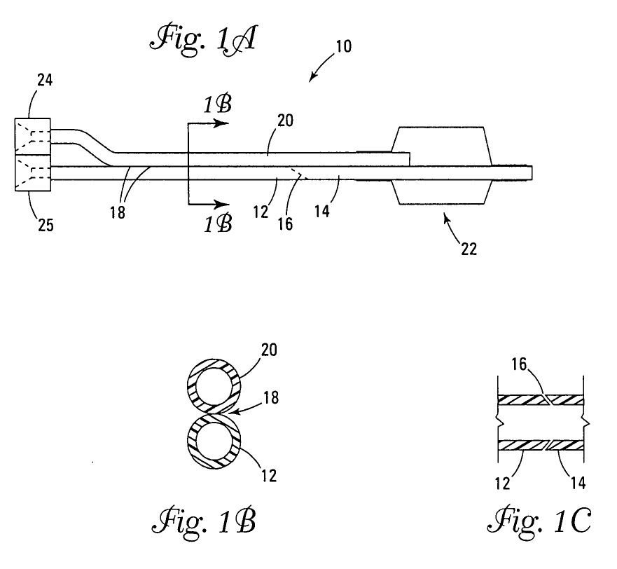

FIGS. 1A and 1B illustrate a balloon catheter 10 of the invention. Removable wire lumen segment 12 is attached to non-removable wire lumen segment 14 by partial precut or tear line 16. Removable wire lumen segment 12 is attached to inflation lumen portion 20 by tear line 18. Balloon 22 (the interventional element) is provided on distal portion of catheter 10. Inflation lumen manifold 24 and guide wire lumen manifold 25 are present on the proximal end of the catheter. The wire lumen segments 12 and 14 and inflation lumen portion 20 are preferably made of co-extruded high density polyethylene.

The catheter 10 can be used in an over-the-wire configuration without any modification. The catheter 10 can be used in a rapid exchange configuration by removing removable wire lumen segment 12. The catheter 10 is preferably used with a guide catheter for coronary application. In peripheral applications, a long sheath is often used in place of a guide catheter.

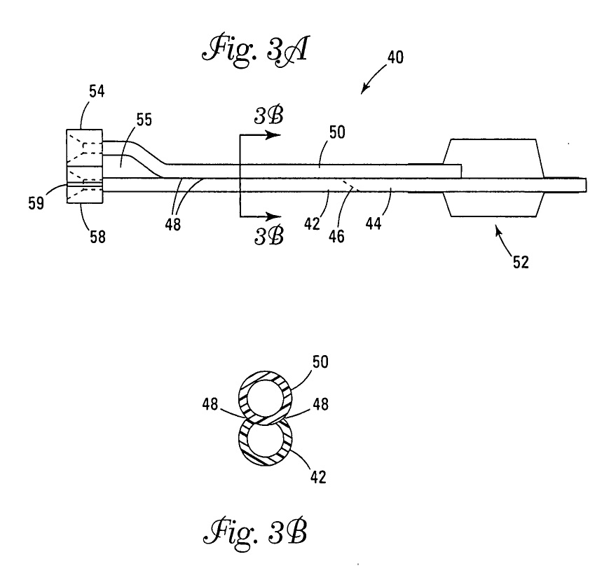

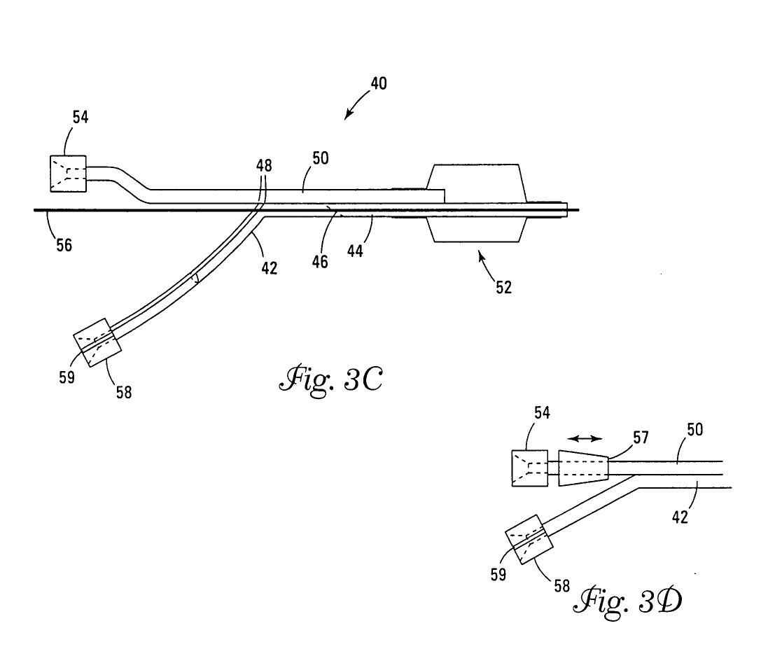

The operator physician can choose the configuration (over-the-wire or rapid exchange) of the catheter in advance or, depending on the specific design chosen, at the time of or prior to the time of withdrawal of the catheter from the body. Conversion of the catheter to rapid exchange after the procedure has begun works best if the removable wire lumen segment opens to expose the guide wire. See FIG. 3C. Then the guide wire can be held while the catheter is withdrawn. It is also possible with some configurations of the invention to remove removable wire lumen segment 12 prior to withdrawing the catheter from the body. This is in-situ conversion to rapid exchange.

To convert the catheter to rapid exchange at the time of withdrawal of catheter 10 from the body, the removable wire lumen segment 12 is separated from the inflation lumen portion 20 while the catheter is drawn out of the body. The tear lines 16 and 18 may be thin attachment points or perforation points or weakened lines of separation to keep the tear force low and consistent. The precut or tear line 16 could be a slit totally separating the removable wire lumen segment 12 from the distal non-removable wire lumen segment 14. The precut or tear line could be comprised of two separate nested structures, an example of which is shown for another balloon catheter in FIG. 1C. The distal separation along precut or tear line 16 of the proximal removable wire lumen segment 12 from the distal non-removable wire lumen segment 14 is shown as a dotted line in FIG. 1A. The distal separation along tear line 16 is shown at an angle to facilitate removal of the catheter from the guide wire without the wire lumen catching on the guide catheter distal end. The optional guide wire lumen manifold 25 may be configured to be axially removable from the removable wire lumen segment 12 or equipped with a tearaway slot or an open slot for the guide wire to exit.

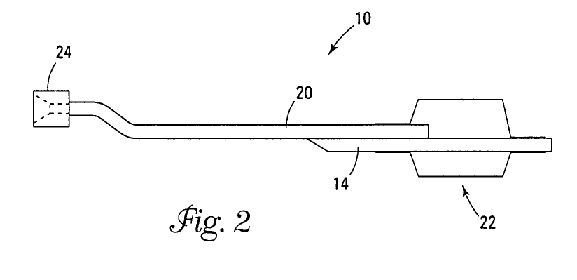

The catheter 10 after the removable wire lumen segment 12 is removed is shown in FIG. 2. Once the removable wire lumen segment 12 is removed, the catheter is a rapid exchange type catheter.

In a preferred embodiment, the separation of the removable wire lumen segment should also open up the wire lumen so the guide wire is free to exit the wire lumen. This allows the guide wire to be held while the wire lumen is torn away or withdrawn axially out of the body as used during a case. This embodiment of the invention is shown in FIGS. 3A to 3D.

FIGS. 3A to 3C illustrate a balloon catheter 40 of the invention. Removable wire lumen segment 42 is attached to non-removable wire lumen segment 44 by tear line 46. Alternatively, tear line 46 may be a partial or complete cut with no attachment axially between removable wire lumen segment 42 and non-removable wire lumen segment 44. Removable wire lumen segment 42 is attached to inflation lumen portion 50 by tear lines 48. Balloon 52 (the interventional element) is provided on the distal portion of the catheter 40. Inflation lumen manifold 54 and guide wire lumen manifold 58 are present on the proximal end of the catheter. The optional guide wire lumen manifold 58 may be configured to be axially removable from the removable wire lumen segment 42 or equipped with a tearaway slot or open slot 59 for the guide wire to exit. The wire lumen segments 42 and 44 and inflation lumen portion 50 are preferably made of co-extruded high density polyethylene.

The catheter 40 can be used in an over-the-wire configuration without any modification. The catheter 40 can be used in a rapid exchange configuration by removing removable wire lumen segment 42. One way to remove the removable wire lumen segment 42 is to hold in one hand both manifolds 54 and 58, then with the other hand inserting any suitable blunt object such as a closed hemostat tip (not shown) into space 55, and then pushing the hemostat tip distally to effect separation of removable wire lumen segment 42 from inflation lumen portion 50. As shown in FIG. 3D, such a separation element may be optionally attached to the distal end of the inflation lumen manifold 54 with a reversible mechanical connection such as a snap connector. By unsnapping the separation element from the manifold, the separation element 57 can slide over the inflation lumen portion 50, separating removable wire lumen segment 42. Alternatively, each manifold 54, 58 can be grasped in a hand and the two manifolds pulled apart to effect separation at tear lines 46 and 48.

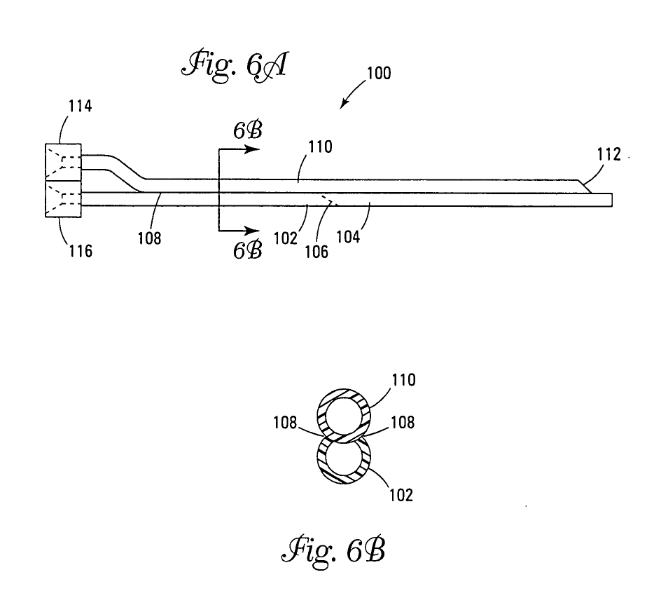

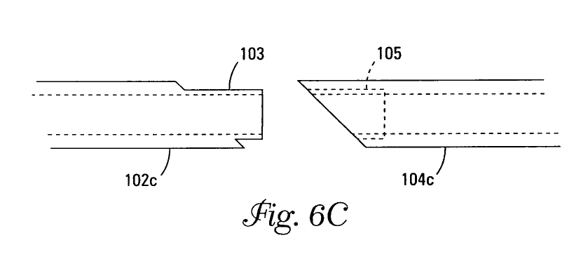

The operator physician can choose the configuration (over-the-wire or rapid exchange) of the catheter in advance or at the time of withdrawal of the catheter 40 from the body. To convert the catheter to rapid exchange at the time of withdrawal of the catheter 40 from the body, the removable wire lumen segment 42 is separated as the inflation lumen portion 50 is drawn out of the body. Specifically, a blunt object or a finger or separation element 57 is placed into space 55 and as the catheter is withdrawn from a previously placed guide catheter lateral force is applied to the removable wire lumen segment 42 to effect separation at tear lines 46 and 48. The tear lines 46 and 48 may be thin attachment points or perforation points or weakened lines of separation to keep the tear force low and consistent. The tear line 46 could be a slit totally separating the removable wire lumen segment 42 from the distal non-removable wire lumen segment 44. The tear line could be comprised of two separate nested structures, an example of which is shown for another balloon catheter in FIG. 1C or FIG. 6C.

The removable wire lumen segment 42 is designed in such a way that the tear lines 48 create a wire lumen that leaves a lumen opening the entire length of the removable portion of the removable wire lumen segment 42 such that a wire may be withdrawn from the lumen through the slit or slot formed by the tear lines 48. This allows the easy conversion to rapid exchange during a procedure when the device started as an over-the-wire catheter with a guide wire in place in the artery through the guide wire lumen. FIG. 3C shows the guide wire 56 and shows catheter 40 as the removable wire lumen segment 42 is being removed.

In an alternative use a guide catheter is placed into a coronary artery as previously described. An over-the-wire length guide wire with frangible joint is advanced past a region of interest. Catheter 40 is advanced in an over-the-wire fashion to or past the region of interest. The guide wire is shortened to rapid exchange length by snapping the wire at the frangible connection, and the catheter 40 is withdrawn and the removable wire lumen segment 42 can be separated in the manner described above.

In yet another alternative use the catheter 40 can be preloaded with a rapid exchange length guide wire and advanced through a guide catheter to a region of interest. The catheter can then be withdrawn and the removable wire lumen segment 42 can be separated in the manner described above.

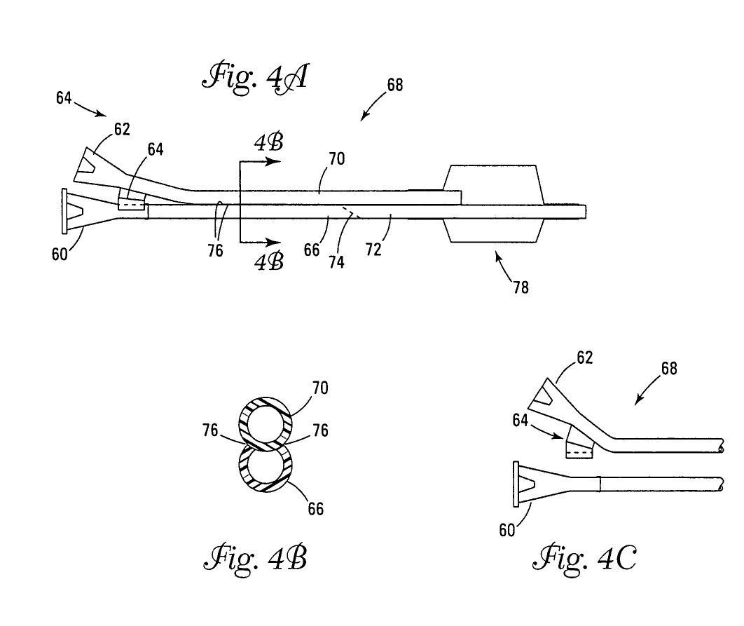

In another balloon catheter 68 of the invention shown in FIGS. 4A to 4C, the guide wire lumen manifold 60 can be clipped into an optional holder 64 on the inflation port 62 to maintain the guide wire lumen segments 66 and 72 in an axial direction for over-the-wire use and keep the guide wire in axial orientation. The guide wire lumen manifold 60 can be withdrawn sideways to initiate the tear away process as in FIGS. 1 to 3 above. The guide wire lumen manifold 60 may be configured to be axially removable from the removable wire lumen segment 66 or equipped with a tearaway slot or open slot for the guide wire to exit.

In FIGS. 4A to 4C, removable wire lumen segment 66 is attached to non-removable wire lumen segment 72 by partial cut or tear line 74. Removable wire lumen segment 66 is attached to inflation lumen portion 70 by tear lines 76. Balloon 78 (the interventional element) is provided on the distal portion of the catheter 68. The wire lumen segments 66 and 72 and inflation lumen portion 70 are preferably made of co-extruded high density polyethylene.

The catheter 68 can be used in an over-the-wire configuration without any modification. The catheter 68 can be used in a rapid exchange configuration by removing removable wire lumen segment 66.

The operator physician can choose the configuration (over-the-wire or rapid exchange) of the catheter in advance or at the time of withdrawal of the catheter 68 from the body. To convert the catheter to rapid exchange at the time of withdrawal of the catheter 68 from the body, the removable wire lumen segment 66 is separated as the inflation lumen portion 70 is drawn out of the body. The tear lines 76 and 74 may be thin attachment points or perforation points or weakened lines of separation to keep the tear force low and consistent. The tear line 74 could be a slit totally separating the removable wire lumen segment 66 from the distal non-removable wire lumen segment 72. The tear line could be comprised of two separate nested structures, an example of which is shown for another balloon catheter in FIG. 1C or FIG. 6C.

The removable wire lumen segment 66 is designed in such a way that the tear lines 76 create a wire lumen that leaves a lumen opening the entire length of the removable portion of the removable wire lumen segment 66 such that a wire may be withdrawn from the lumen through the slit or slot formed by the tear lines 76. This allows the easy conversion to rapid exchange during a procedure when the device started as an over-the-wire catheter with a guide wire in place in the artery through the guide wire lumen. In FIG. 4C, the tear away process has already begun, and the guide wire lumen manifold 60 is shown separated from the holder 64.

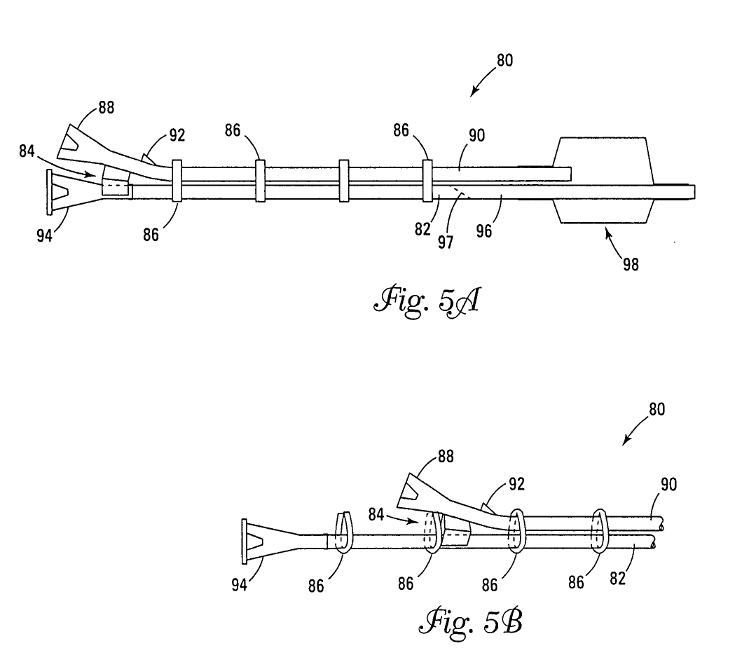

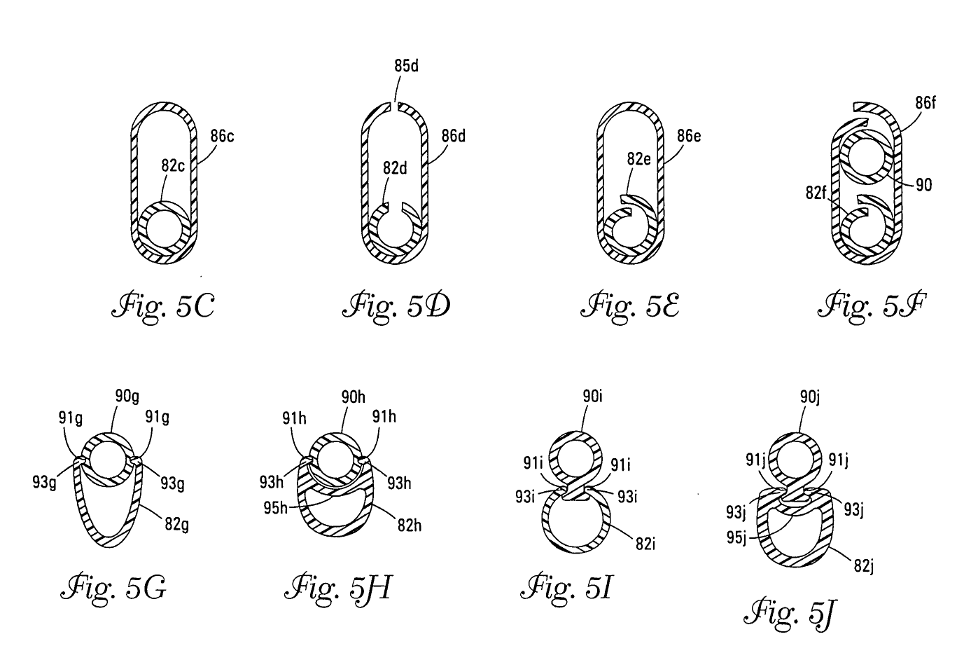

In another catheter 80 of the invention shown in FIGS. 5A to 5J, the removable wire lumen segment 82 may be removed by axially pulling the removable wire lumen segment 82 back proximally (see FIG. 5B). The guide wire lumen manifold 94 can be clipped into a holder 84 on the inflation port 88. The guide wire lumen manifold 94 may be configured to be axially removable from the removable wire lumen segment 82 or equipped with a tearaway slot for the guide wire to exit.

The removable wire lumen segment 82 is attached to the main shaft or inflation lumen portion 90 by one or more bands 86 that allow axial movement and are spaced apart along the shaft, are affixed to the removable wire lumen segment 82, and encircle the main shaft 90 in a sliding fit. Alternatively, a single band can be used over substantially the length of the removable segment, optionally in combination with one or more narrow band 86. Such bands 86 may be torn apart by engagement with the main shaft manifold 88 or cut apart by the manifold. Sharp blade or edge of plastic 92 urges the bands to be cut or torn to release the removable wire lumen segment 82. In this embodiment, the removable wire lumen segment 82 is an extruded tube connected to the main shaft only by one or more bands 86. In a preferred embodiment, the tear line 97 is a complete cut allowing removable wire lumen segment 82 to be axially moveable relative to non-removable wire lumen segment 96. The removable wire lumen segment 82 could be longitudinally preslit (FIGS. 5D to 5F) if desired for guide wire exit or not slit longitudinally (FIG. 5C). A guard, not shown, could be placed over the sharp blade 92 to prevent injury to the operator. Optionally, inflation lumen portion 90 can be placed over a hyptotube so in rapid exchange use the shaft has added support, or one or more wires can be embedded into the wall of the inflation lumen portion 90 or placed in the lumen of the inflation lumen portion 90. Alternatively, the hypotube may comprise the inflation lumen portion 90 and be the main catheter shaft mechanical support element.

In FIG. 5A, removable wire lumen segment 82 is attached to non-removable wire lumen segment 96 by tear line 97. Tear line 97 may alternatively be a complete cut or partial cut to reduce resistance to axial movement. The tear line can also be a nested structure such as that shown in FIG. 1C. The balloon 98 (the interventional element) is provided on the distal portion of the catheter 80. The catheter 80 can be used in an over-the-wire configuration without any modification. The catheter 80 can be used in a rapid exchange configuration by removing removable wire lumen segment 82.

The operator physician can choose the configuration (over-the-wire or rapid exchange) of the catheter in advance or at the time of withdrawal of the catheter 80 from the body. To convert the catheter to rapid exchange at the time of withdrawal of the catheter 80 from the body, the removable wire lumen segment 82 is separated by pulling removable wire lumen segment 82 proximally while holding back the catheter 90. Alternatively, to convert the catheter to rapid exchange prior to use of the catheter, the removable wire lumen segment 82 is pulled proximally a few centimeters, exposing the proximal opening of the non-removable wire lumen segment 96 at tear away 97. In this embodiment it is not necessary to separate the removable wire lumen segment 82.

FIG. 5C shows band 86c and removable wire lumen segment 82c. FIG. 5D shows band 86d having a slit or slot 85d and removable wire lumen segment 82d having a slit or slot. FIG. 5E shows band 86e and removable wire lumen segment 82e having an overlapping slit. Inflation lumen portion 90 is not shown in FIGS. 5C to 5E. FIG. 5F shows inflation lumen portion 90, removable wire lumen segment 82f having an overlapping slit, and band 86f having an overlapping slit. It is contemplated that a hypotube may be used for the inflation lumen portion 90 in all embodiments shown in FIG. 5C to 5F.

As an alternative to bands, an interlocking sliding rail arrangement may be used to connect inflation lumen portion 90 to removable wire lumen segment 82 as shown in FIGS. 5G to 5J. In FIG. 5G, inflation lumen portion 90g is provided with longitudinal grooves 91g which cooperate with ears 93g on removable wire lumen segment 82g to effect a longitudinally slidable connection that resists radial separation forces. In this embodiment, removable wire lumen segment 82g is preferably made of polyethylene or nylon but may be made of other polymers as well. It is contemplated that a hypotube may be used for the inflation lumen portion 90 in all embodiments shown in FIG. 5G to 5J. Preferably, removable wire lumen segment 82g is preloaded to provide compressive force on ears 93g into longitudinal grooves 91g. Optionally, removable wire lumen segment 82h can be provided with web 95h as shown in FIG. 5H. Web 95h can help prevent disengagement of ears 93h from slots 91h by providing resistance to separation of ears 93h. Further, web 95h can help maintain engagement of ears 93h in slots 91h by preventing inflation lumen portion 90h from slipping sideways into the lumen of removable wire lumen segment 82h.

Another embodiment of an interlocking sliding rail arrangement is shown in FIGS. 5I and 5J. In FIG. 5I, inflation lumen portion 90i is provided with a rail portion having longitudinal grooves 91i. Removable wire lumen segment 82i is provided with ears 93i which cooperate with grooves 91i to effect a longitudinally slidable connection that resists radial separation forces. In this embodiment it may not be necessary to provide a compressively pre-loaded connection between ears 93i and grooves 91i. Optionally, removable wire lumen segment 82j can be provided with web 95j as shown in FIG. 5J. Web 95j can help prevent disengagement of ears 93j from slots 91j by providing resistance to separation of ears 93j.

FIGS. 6A and 6B illustrate an infusion/dye-injection/suction catheter 100 of the invention. Removable wire lumen segment 102 is attached to non-removable wire lumen segment 104 by tear line 106. Removable wire lumen segment 102 is attached to infusion/dye-injection/suction lumen portion 110 by tear lines 108. As shown in FIG. 6C, optionally, removable wire lumen segment 102c is manufactured separately from lumen 110. Tear line or precut 106 can be a complete cut end of removable wire lumen segment 102c, with the distal end of removable wire lumen segment 102c piloting into the proximal end of non-removable wire lumen segment 104c. Pilot 103 pilots into pilot holder 105. The infusion/dye-injection/suction port 112 (the interventional element) is provided on the distal portion of the catheter 100. Infusion/dye-injection/suction lumen manifold 114 and guide wire lumen manifold 116 are present on the proximal end of the catheter. The optional guide wire lumen manifold 116 may be configured to be axially removable from the removable wire lumen segment 102 or equipped with a tearaway slot or open slot for the guide wire to exit. The wire lumen segments 102 and 104 and infusion/dye-injection/suction lumen portion 110 are preferably made of co-extruded high density polyethylene.

The catheter 100 can be used in an over-the-wire configuration without any modification. The catheter 100 can be used in a rapid exchange configuration by removing removable wire lumen segment 102. The operator physician can choose the configuration (over-the-wire or rapid exchange) of the catheter in advance or at the time of withdrawal of the catheter 100 from the body. To convert the catheter to rapid exchange at the time of withdrawal of the catheter 100 from the body, the removable wire lumen segment 102 is separated as the infusion/dye-injection/suction lumen portion 110 is drawn out of the body. The tear lines 106 and 108 may be thin attachment points or perforation points or weakened lines of separation to keep the tear force low and consistent. The tear line 106 could be a slit totally separating the removable wire lumen segment 102 from the distal non-removable wire lumen segment 104. The tear line could be comprised of two separate nested structures, an example of which is shown for another catheter in FIG. 1C or FIG. 6C.

The distal separation along tear line 106 of the proximal removable wire lumen segment 102 from the distal non-removable wire lumen segment 104 is shown as a dotted line in FIG. 6A. The distal separation along tear line 106 is shown at an angle to facilitate removal of the catheter from the guide wire without the wire lumen catching on the guide catheter distal end.

The removable wire lumen segment 102 is designed in such a way that the tear lines 108 create a wire lumen that leaves a lumen opening the entire length of the removable portion of the removable wire lumen segment 102 such that a wire may be withdrawn from the lumen through the slit or slot formed by the tear lines 108. This allows the easy conversion to rapid exchange during a procedure when the device started as an over-the-wire catheter with a guide wire in place in the artery through the guide wire lumen.

FIGS. 7A and 7B show cross-sectional views of alternative embodiments of the balloon catheter of FIG. 3. In FIG. 7A, removable wire lumen segment 124 is attached to inflation lumen portion 120 by tear lines 126. Inflation lumen portion 120 is reinforced by hypotube 122. In FIG. 7B, removable wire lumen segment 134 is attached to inflation lumen portion 130 by tear lines 136. Inflation lumen portion 130 is reinforced by wires 132. In FIG. 7C, removable wire lumen segment 144 is attached to inflation lumen portion 140 by tear lines 146. Inflation lumen portion 140 is reinforced by wire 142 disposed in the inflation lumen.

In FIG. 7D, removable wire lumen segment 124 is attached to inflation lumen surrounding portion 121. Inflation lumen surrounding portion 121 is reinforced by hypotube 123. Hypotube 123 serves as the conduit for the fluid to control inflation or deflation of the balloon. Tear line 125 on inflation lumen surrounding portion 121 allows exit of the hypotube 123. Only the hypotube 123 remains in the proximal portion of the catheter.

In FIG. 7E, removable wire lumen segment 124e is attached to inflation lumen surrounding portion 121e. Inflation lumen surrounding portion 121e is reinforced by hypotube 123e. Hypotube 123e serves as the conduit for the fluid to control inflation or deflation of the balloon. Precut slot or slit 125e on inflation lumen surrounding portion 121e allows exit of the hypotube 123e. Only the hypotube 123e remains in the proximal portion of the catheter. Removable wire lumen segment 124e and inflation lumen surrounding portion 121e are fabricated as a unitary structure in a modified figure eight cross section. Gap 127e in unitary structure permits guide wire (not shown) to exit the removable wire lumen segment 124e in a manner similar to that described in connection with FIGS. 3A to 3D.

In FIG. 7F, removable wire lumen segment 124f is attached to inflation lumen surrounding portion 120f by a single linear attachment line 146f. Inflation lumen surrounding portion 120f is reinforced by hypotube 122f. Hypotube 122f serves as the conduit for the fluid to control inflation or deflation of the balloon. Tear line 125f on inflation lumen surrounding portion 120f allows exit of the hypotube 122f. Only the hypotube 122f remains in the proximal portion of the catheter. The tear line between the removable wire lumen segment 124f and the non-removable wire lumen segment extends to include inflation lumen surrounding portion 120f.

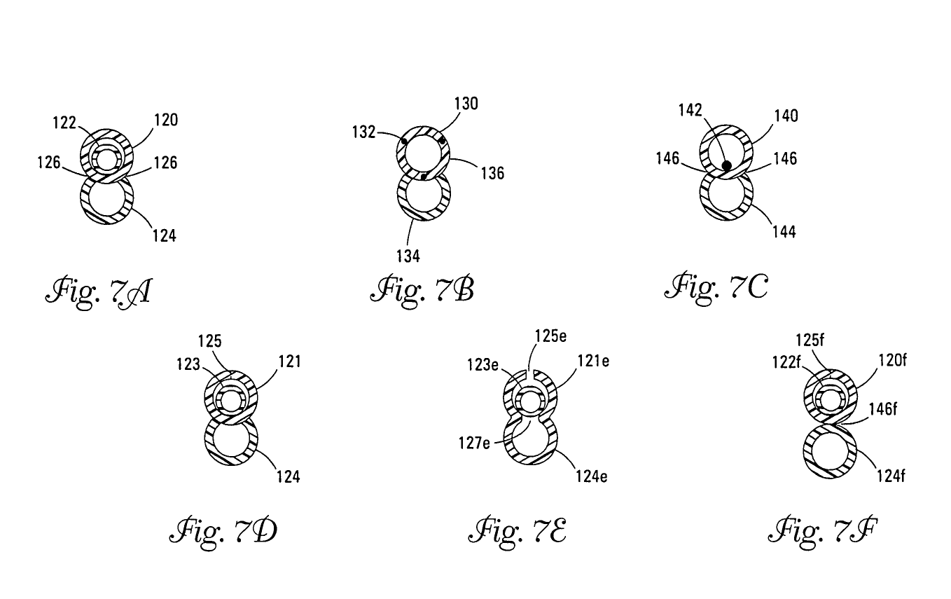

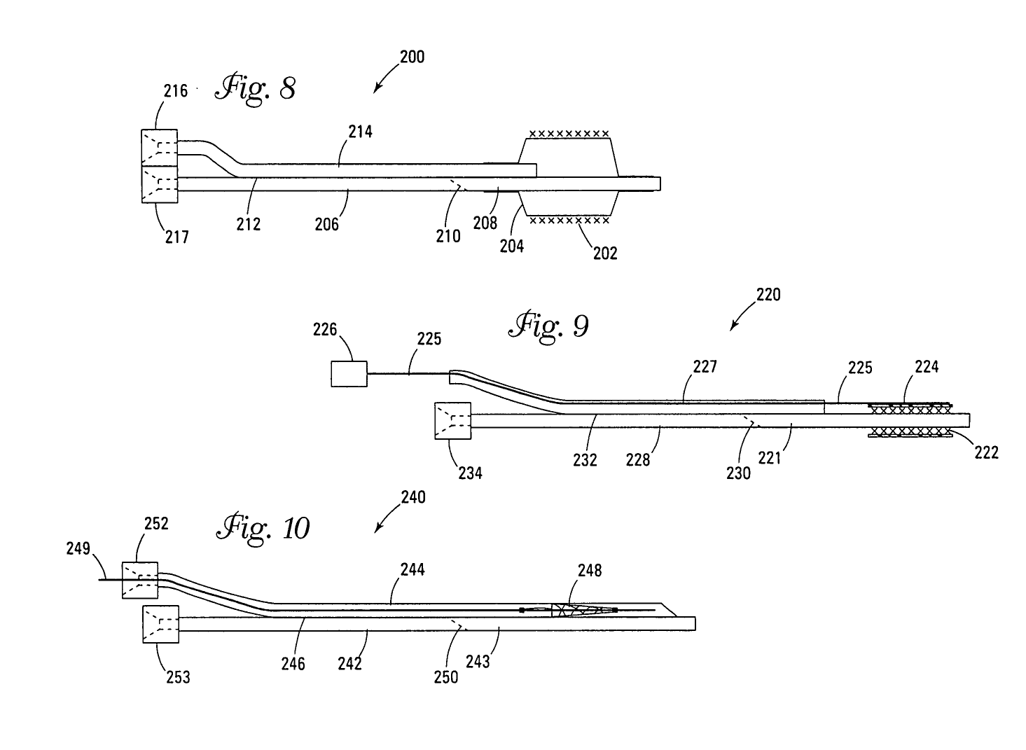

FIG. 8 shows a stent delivery catheter 200 of the present invention. Balloon expandable stent 202 is shown over balloon 204. Balloon 204 and stent 202 are shown in an expanded state. Stent delivery catheter 200 and its use are similar in many respects to catheter 40 of FIG. 3.

In FIG. 8, removable wire lumen segment 206 is attached to non-removable wire lumen segment 208 by tear line 210. Removable wire lumen segment 206 is attached to inflation lumen portion 214 by tear lines 212. The balloon 204 and the stent 202 (together forming the interventional element) are provided on the distal portion of the catheter 200. Inflation lumen manifold 216 and guide wire lumen manifold 217 are present on the proximal end of the catheter. The optional guide wire lumen manifold 217 may be configured to be axially removable from the removable wire lumen segment 206 or equipped with a tearaway slot or open slot for the guide wire to exit. The wire lumen segments 206 and 208 and inflation lumen portion 214 are preferably made of co-extruded high density polyethylene. The catheter 200 can be used in an over-the-wire configuration without any modification. The catheter 200 can be used in a rapid exchange configuration by removing removable wire lumen segment 206.

FIG. 9 shows a stent delivery catheter 220 of the present invention. Self expandable stent 222 is shown over non-removable wire segment 221 and within sheath 224. Stent 222 is shown in a contracted state. Sheath 224 is attached to control wire 225 to which is attached handle 226. Control wire 225 passes within lumen of control tube 227 (the delivery element). Stent delivery catheter 220 and its use are similar in many respects to catheter 40 of FIG. 3.

In FIG. 9, removable wire lumen segment 228 is attached to non-removable wire lumen segment 221 by tear line 230. Removable wire lumen segment 228 is attached to control tube 227 by tear lines 232. The stent 222 (the interventional element) is provided on the distal portion of the catheter 220. A guide wire lumen manifold 234 is present on the proximal end of the catheter. The optional guide wire lumen manifold 234 may be configured to be axially removable from the removable wire lumen segment 228 or equipped with a tearaway slot or open slot for the guide wire to exit.

The catheter 220 can be used in an over-the-wire configuration without any modification. The catheter 220 can be used in a rapid exchange configuration by removing removable wire lumen segment 228.

FIG. 10 shows an embolic protection device delivery catheter 240 of the present invention. The device of FIG. 10 has some similarities to the device of FIG. 6. Removable wire lumen segment 242 is attached to device delivery lumen portion 244 by tear lines 246. Embolic protection device 248 is slideably received in device delivery lumen portion 244 and host wire 249 of embolic protection device 248 exits from device delivery lumen portion 244.

Removable wire lumen segment 242 is attached to non-removable wire lumen segment 243 by tear line 250. Device delivery lumen manifold 252 and guide wire lumen manifold 253 are present on the proximal end of the catheter. The optional guide wire lumen manifold 253 may be configured to be axially removable from the removable wire lumen segment 242 or equipped with a tearaway slot or an open slot for the guide wire to exit. The catheter 240 can be used in an over-the-wire configuration without any modification. The catheter 240 can be used in a rapid exchange configuration by removing removable wire lumen segment 242.

Many interventional devices could be used with the catheter of FIG. 10, such as flow measuring guide wires, intravascular cooling catheters, cryogenic vessel wall treatment catheters, and others. Further, implants could be used with the catheter of FIG. 10, such as temporary vena cava filters with tethers. Non-tethered percutaneously delivered implants can be used with the catheter of FIG. 10, such as mitral valve annuloplasty devices, atrial appendage closure or sealing devices, septal defect closure devices, and the like. Non-tethered implants can be pre-loaded into the catheter at the factory, for example, and delivered from the catheter lumen by means of a push rod or other equivalent structure. The non-tethered implants may be reversibly attached to the push rod. It is contemplated that devices or implants can be delivered or recovered with the catheter of FIG. 10.

The catheter of FIG. 10 can be used as follows. A guide catheter is placed in a coronary ostium as described earlier. A guide wire is advanced within the guide catheter to a region of interest within the coronary vessel. The guide wire is back loaded into the tip of non-removable catheter segment 243 and catheter 240 is advanced over the guide wire to a vicinity of the region of interest. Embolic protection device 248 is loaded into and advanced distally through device delivery lumen portion 244. Alternatively, embolic protection device 248 could be preloaded into device delivery lumen portion 244. Embolic protection device 248 is advanced out of device delivery lumen portion 244 and into vessel. Alternatively catheter 240 can be withdrawn to expose embolic protection device 248. Catheter 240 is removed from vessel, preferably in a manner that separates removable wire lumen segment 242 from device delivery lumen portion 244, similar to that previously described. Embolic protection device 248 remains in the vessel to capture emboli.

At the time it is desired to recover embolic protection device 248 with captured emboli retained therein, host wire 249 is back loaded into distal tip of non-removable wire segment 243 and catheter 240 is advanced over host wire 249 until catheter 240 tip reaches embolic protection device 248. At this point embolic protection device 248 can be withdrawn into catheter 240 or the catheter can be advanced over embolic protection device 248. Catheter 240 with embolic protection device 248 therein can then be withdrawn from the patient's body.

The catheter of FIG. 10 can be used in a similar fashion for delivery of other percutaneous devices such as occlusive devices for the left atrial appendage of the heart, occlusive devices for cardiac septal defects such as ASD (atrial septal defects), PFO (patent foramen ovale), and PDA (patent ductus arteriosus), for devices designed to remodel the mitral valve, and others. The catheter of FIG. 10 can also be used for recovery of percutaneous devices such as those listed above. The catheter can be used to deliver devices into coronary arteries or veins, the coronary sinus, peripheral or neurological arteries or veins, to chambers in the heart, and elsewhere as will be apparent to those skilled in the art.

The above description and the drawings are provided for the purpose of describing embodiments of the invention and are not intended to limit the scope of the invention in any way. It will be apparent to those skilled in the art that various modifications and variations can be made without departing from the spirit or scope of the invention. Thus, it is intended that the present invention cover the modifications and variations of this invention provided they come within the scope of the appended claims and their equivalents.

1. A catheter for use in combination with an elongate support member, the catheter comprising:
an elongate body having a proximal portion, a proximal end, a distal portion, a distal end, and a main shaft;
an element disposed on the distal portion of the elongate body, the element being an interventional element or a delivery element for delivery of an interventional element;
at least one lumen dimensioned to receive the elongate support member; and
a tube wall disposed about the lumen,
wherein the tube wall has at least one non-removable segment disposed on the distal portion of the elongate body and at least one removable segment,
wherein the removable segment is tubular or generally tubular having a partial circular circumferential cross section,
wherein the transverse cross-sectional area of the removable segment does not comprise the entire transverse cross-sectional area of the catheter,
wherein the removable segment can be completely removed from the catheter, and
wherein at least a portion of the removable segment is disposed on a proximal portion of the elongate body, the proximal portion of the elongate body being that portion of the elongate body from the proximal end to immediately before a midpoint between the proximal end and the distal end of the elongate body.

2. A catheter of claim 1, wherein the tube wall has a single removable segment disposed on the proximal portion of the elongate body.

3. A catheter of claim 1, wherein the removable segment is tubular.

4. A catheter of claim 1, wherein the removable segment is generally tubular having a partial circular cross section.

5. A catheter of claim 1, wherein the at least one removable segment has a longitudinal slit through its entire length.

6. A catheter of claim 1, wherein the tube wall extends from the proximal end to the distal end of the elongate body.

7. A catheter of claim 1, wherein the at least one removable segment of the tube wall is adjacent to the non-removable segment and is attached to the non-removable segment by a tear line.

8. A catheter of claim 7, wherein the tear line between the at least one removable segment of the tube wall and the non-removable segment of the tube wall is selected from thin attachment points, perforation points, a weakened line of separation, or a slit.

9. A catheter of claim 8, wherein the tear line between the at least one removable segment of the tube wall and the non-removable segment of the tube wall is a weakened line of separation.

10. A catheter of claim 8, wherein the tear line between the at least one removable segment of the tube wall and the non-removable segment of the tube wall is a slit.

11. A catheter of claim 10, wherein the slit is formed by the distal end of the removable segment and the proximal end of the non-removable segment, and the distal end of the removable segment and the proximal end of the non-removable segment are nested.

12. A catheter of claim 7, wherein the at least one removable segment of the tube wall is attached to the main shaft of the catheter by one or more tear lines.

13. A catheter of claim 12, wherein the one or more tear lines between the at least one removable segment of the tube wall and the main shaft of the catheter are selected from thin attachment points, perforation points, or one or more weakened lines of separation.

14. A catheter of claim 12, wherein the catheter has two tear lines between the at least one removable segment of the tube wall and the main shaft and the removable segment is generally tubular having a partial circular cross section.

15. A catheter of claim 1, wherein the at least one removable segment of the tube wall is attached to the main shaft of the catheter by breakable bands.

16. A catheter of claim 15, wherein the breakable bands are circular and are fixedly attached to the at least one removable segment and not fixedly attached to the main shaft.

17. A catheter of claim 1, wherein the at least one removable segment of the tube wall is attached to the main shaft of the catheter by an interlocking sliding rail arrangement.

18. A catheter of claim 1, wherein the main shaft comprises a main shaft lumen and the main shaft lumen has a main shaft tube wall disposed about the lumen.

19. A catheter of claim 18, wherein the tube wall and the main shaft tube wall are formed from a co-extruded polymer.

20. A catheter of claim 19, wherein the polymer is high density polyethylene.

21-51. (canceled)
